(19) United States
(12) Patent Application Publication
(10) Pub. No.: US 2019/0034862 A1
(43) Pub. Date: Jan. 31, 2019

(54) Inventory Control System

(71) Applicant: Mylan Inc., Canonsburg, PA (US)

(72) Inventors: Eric W. Starr, Butler, PA (US); Molly Knewtson, Pittsburgh, PA (US)

(73) Assignee: Mylan Inc., Canonsburg, PA (US)

(21) Appl. No.: 16/004,488

(22) Filed: Jun. 11, 2018

Related U.S. Application Data
(60) Provisional application No. 62/518,368, filed on Jun. 12, 2017.

(51) Int. Cl. G06Q 10/08; G16H 20/13

(52) U.S. Cl. CPC G06Q 10/087; G16H 20/13

(57) ABSTRACT

An inventory control system includes an inventory control hub and a medicament container in communication with the inventory control hub and configured to exchange medicament information with the inventory control hub, the medicament container including a body defining a container cavity; a plurality of medicament receptacles, each medicament receptacle configured to accommodate removable storage of an unused auto-injector within the medicament receptacle and prevent storage of a used auto-injector within the medicament receptacle; and a presence sensor for each medicament receptacle, each presence sensor configured to detect a presence or absence of the unused auto-injector from the corresponding medicament receptacle.

### FIELD OF THE INVENTION

This application relates to an inventory control system for medicaments stored in a container, and more particularly relates to an inventory control system for injector medicament devices stored in a container.

### BACKGROUND

Some people suffer from medical conditions that often require immediate treatment to treat the sudden onset of symptoms. In such cases, the patients are often prescribed with particular medicaments to address the sudden symptoms. For example, some people suffer from severe allergies, which may result in anaphylaxis. In such cases, anaphylaxis may be treated through the administration of epinephrine, and patients are often prescribed an auto-injector of epinephrine to treat sudden anaphylaxis.

While patients suffering from such medical conditions are often careful about keeping the particular medicament near them at all times, invariably circumstances arise where the patient is without the medicament. For example, a patient may leave their medicament at home, the patient may be out of the medicament and waiting for a refill, the medicament may have expired, etc. In such cases, these patients risk being in an emergency situation where the symptoms of the medical condition appear but the patient has no medicament to treat the symptoms.

### BRIEF DESCRIPTION OF THE DRAWINGS

The features and components of the following figures are illustrated to emphasize the general principles of the present disclosure. Corresponding features and components throughout the figures can be designated by matching reference characters for the sake of consistency and clarity.

FIG. 1 illustrates an example of an inventory control system according to aspects of the present disclosure.

FIG. 2 illustrates a container of the inventory control system of FIG. 1.

FIG. 3 illustrates the container of FIG. 2 with a cover of the container removed.

FIG. 4A illustrates a used auto-injector.

FIG. 4B illustrates an unused auto-injector.

FIG. 5 illustrates an exemplary hardware diagram for the container of FIG. 1.

### DETAILED DESCRIPTION

The subject matter of examples of the present invention is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

In various examples, an inventory control system includes an inventory control hub and a medicament container in communication with the inventory control hub. In some examples, the medicament container includes a body defining a container cavity and a plurality of medicament receptacles. Each medicament receptacle is configured to accommodate removable storage of an unused auto-injector within the medicament receptacle and to prevent storage of a used auto-injector within the medicament receptacle. According to some examples, a presence sensor is provided for each medicament receptacle. Each presence sensor is configured to detect a presence or absence of the unused auto-injector from the corresponding medicament receptacle. In various examples, each presence sensor is positioned within the corresponding medicament receptacle.

In some examples, the medicament container includes a controller that is in communication with each presence sensor.

According to certain examples, the controller is configured to remain in a low power state for predetermined time intervals, and after each predetermined time interval, the controller is configured to: exit the low power state; receive sensor data from each presence sensor; determine medicament inventory level information based on the sensor data; transmit the medicament inventory level information to the inventory control hub; and return to the low power state and start another predetermined time interval.

According to some examples, the controller is configured to: enter a low power state; exit the low power state if at least one of the presence sensors detects an absence of an unused auto-injectors from the corresponding medicament receptacle; transmit medicament inventory level information to the inventory control hub based on the absence of the unused auto-injector; and return to the low power state.

In certain examples, the presence sensors are selected from the group including, but not limited to, mechanical switches, photoelectric sensors, capacitance sensors, optical sensors, pressure sensors, weight sensors, and ultrasonic sensors.

According to various aspects, the medicament container includes at least one of a transmission button, an on-demand communication component, a replaceable power source, a visual indicator, or a holder for each medicament receptacle. The transmission button is configured to cause the medicament container to transmit medicament inventory level information to the inventory control hub when selected. The on-demand communication component is configured to enable real-time communication with the inventory control hub. In some examples, the replaceable power source is a battery, and the visual indicator indicates a status of the battery (or other replaceable power source).

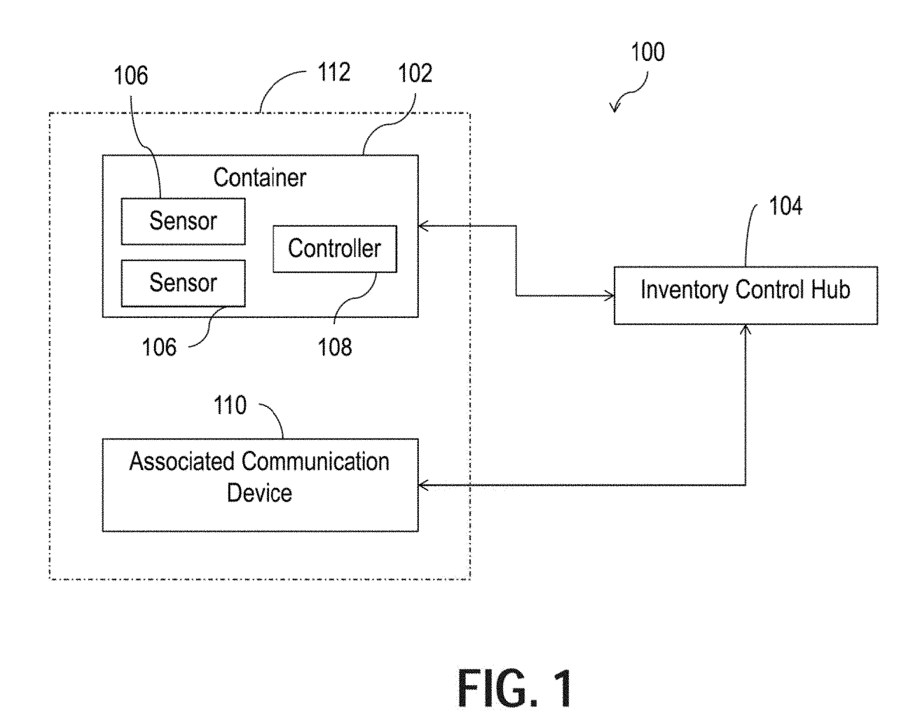

FIG. 1 illustrates an example of an inventory control system 100. The inventory control system 100 includes a container 102 for medicament storage and an inventory control hub 104. The container 102 includes multiple sensors 106 that are provided to detect the presence or absence of a medicament device within the container 102, and a controller 108 for processing the sensor data. As described below, during various inventory control processes, the container 102 communicates medicament inventory information to the inventory control hub 104. In some cases and as described below, the inventory control hub 104 communicates with a communication device 110 associated with the location 112 of the container 102 in response to the transmission of the medicament inventory information to the inventory control hub 104.

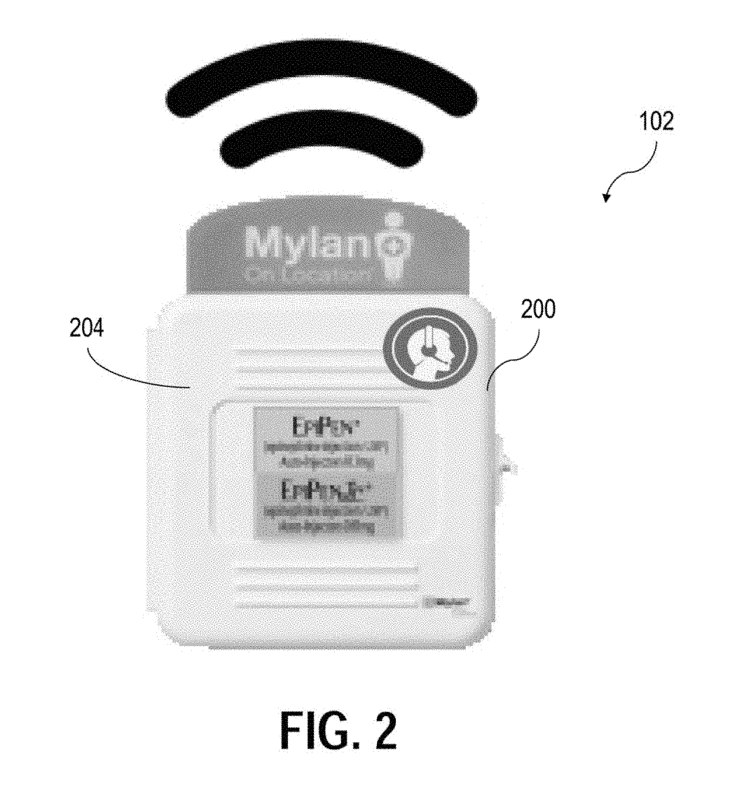

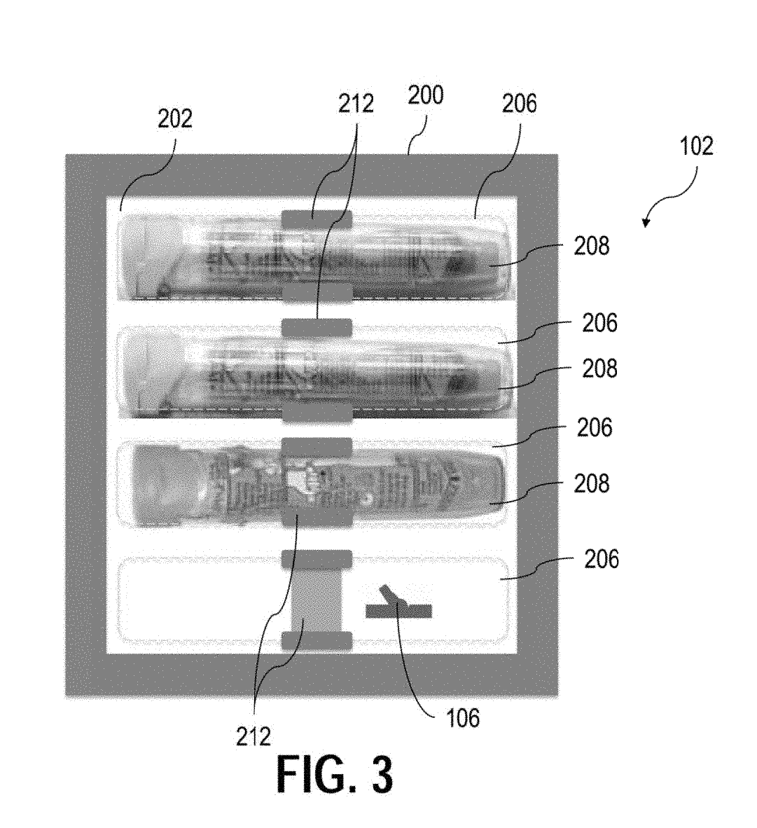

FIGS. 2 and 3 illustrate the container 102. The container 102 includes a body 200 that defines a container cavity 202 (see FIG. 3). A cover 204 is connected to the body 200 and provides access to the container cavity 202. In various examples, the container 102 is configured to remain at the predetermined location 112. As one non-limiting example, the container 102 may be mounted on a post, wall or other surface, or other suitable locations at the predetermined location 112. In other examples, the container 102 may be portable, either within the location 112 or external to the location 112.

As illustrated in FIG. 3, the container cavity 202 includes multiple medicament receptacles 206 that are sized to accommodate unused auto-injectors 208. Although reference is made to auto-injectors 208, various other types of medicament devices may be provided with the container 102. In the present example, the container cavity 202 includes four medicament receptacles 206 with an auto-injector 208 removed from one of the medicament receptacles 206. It will be appreciated that the number of medicament receptacles 206 should not be considered limiting on the current disclosure. In various examples, the container cavity 202 includes at least two medicament receptacles 206. In some examples, the medicament receptacles 206 may be integrally formed with the body 200 (such as through injection molding or other suitable forming techniques). In other examples, the medicament receptacles 206 may be an insert secured within the container cavity 202 through various suitable securing mechanisms, including, but not limited to, various mechanical fasteners or chemical agents.

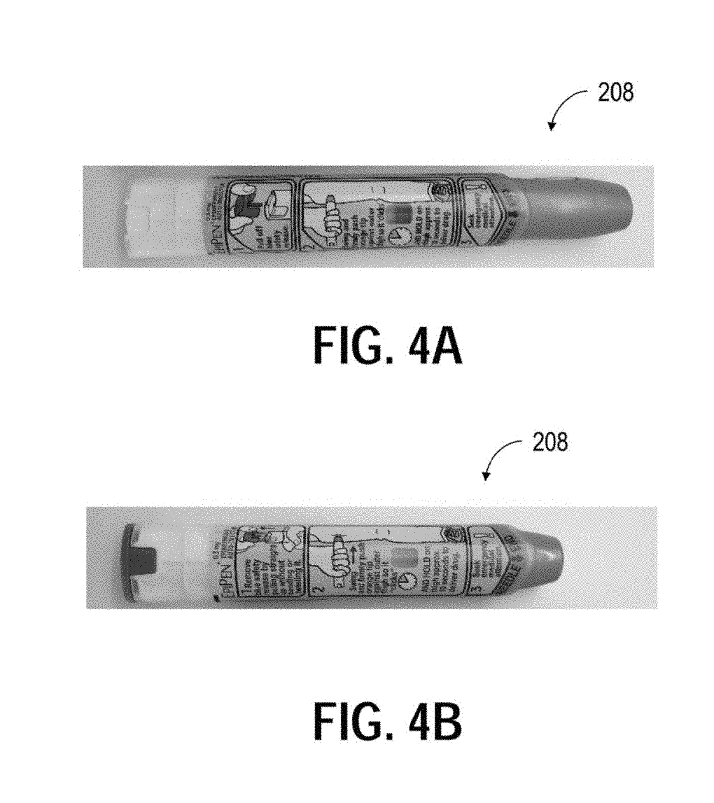

The medicament receptacles 206 are dimensioned such that they can only accommodate unused auto-injectors 208 (i.e., they cannot accept used auto-injectors 208). As illustrated in FIGS. 4A-B, the shape of the auto-injector changes depending on whether the auto-injectors has been used (FIG. 4A) or unused (FIG. 4B). In some cases, a length of the auto-injector 208 increases after it has been used. For example, the length of the used auto-injector 208 in FIG. 4A is greater than the length of the unused auto-injector 208 in FIG. 4B. Accordingly, because the medicament receptacles 206 are dimensioned to only accommodate unused auto-injectors 208, an auto-injector 208 may not be re-inserted into one of the medicament receptacles 206 after the auto-injector 108 has been removed from the container 102.

In some examples, each medicament receptacle 206 optionally includes a holder 212 to aid in retaining the unused auto-injectors 208 within their respective receptacles 206. The holder 212 may include one or more suitable temporary retaining mechanisms including, but not limited to, various gripping materials, pins, hooks, snap-fitting, elastic bands, hook and loop fasteners, etc.

As illustrated in FIG. 3, the sensors 106 are provided within the medicament receptacles 206 to detect the presence or absence of the auto-injector 208 from the particular medicament receptacle 206. In various other examples, the sensors 106 may be positioned at various other locations suitable for detecting the presence or absence of the auto-injector 208, and thus may not be within the medicament receptacles 206. The sensors 106 may be various sensors suitable for detecting the presence of the auto-injector 208 within the medicament receptacle 206, including, but not limited to, mechanical switches, photoelectric sensors, capacitance sensors, optical sensors, pressure sensors, weight sensors, ultrasonic sensors, etc. The sensors 106 are in wired or wireless communication with the controller 108 of the container 102.

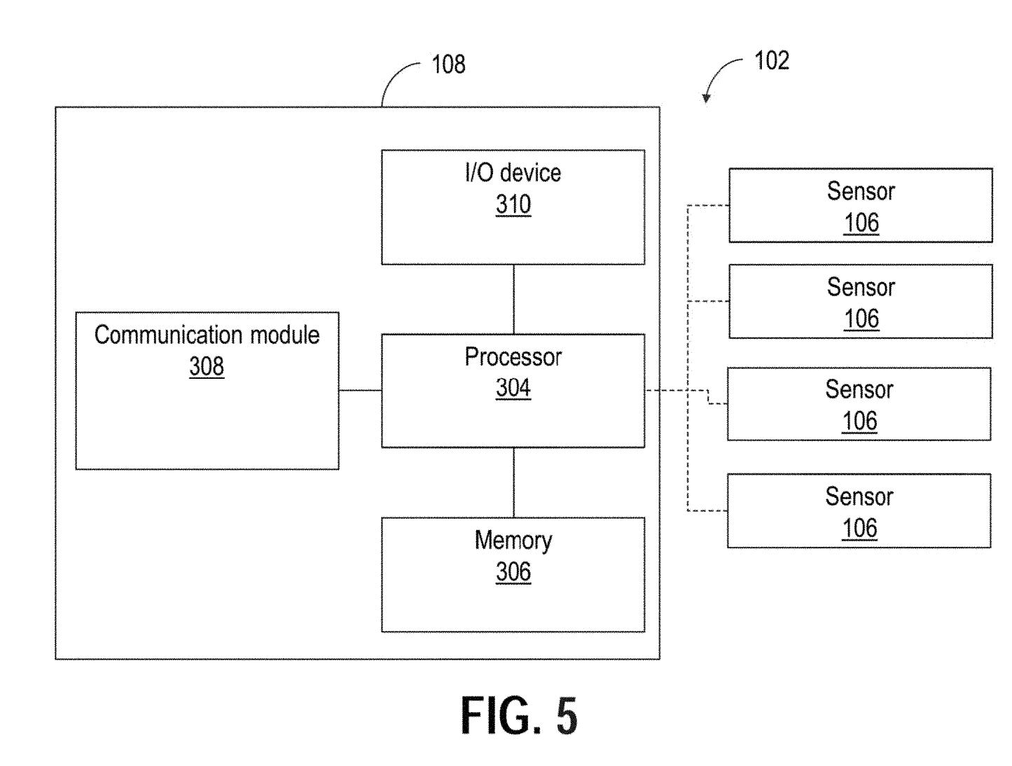

FIG. 5 is a schematic of some of the hardware of the container 102. As previously described, the container 102 includes the sensors 106 in communication with the controller 108. The controller 108 may be provided at any suitable location on or within the container 102. The controller 108 includes a processor 304, which may be a general-purpose computer, special purpose computer, and/or other programmable data processing apparatus, and a memory 306, which stores various instructions that may be executed by the processor 304. The memory 306 is a computer-usable or computer-readable medium that may be any non-transitory medium that is not a transitory signal and can contain or store the program or instructions for use by or in connection with the instruction or execution of a system, apparatus, or device. The controller 108 also includes a communication module 308, which includes the components necessary for the container 102 to establish wired or wireless communication (e.g., cellular communication, Wi-Fi, Bluetooth, etc.) with the inventory control hub 104 via an input/output device 310.

Optionally, the container 102 includes additional features. For example, in some cases, the container 102 includes location detection capabilities (including but not limited to global positioning systems) such that the container 102 may ascertain its particular location. In some examples, the location information of the container 102 may be provided as part of the data transmitted from the container 102 to the inventory control hub 104. The container 102 may also include replaceable batteries, although various other suitable power sources may be used. In some of these examples, various indicators such as lights or other indicators may be provided on the container 102 to indicate a status of the power supply.

In some examples, the container 102 has a unique identification, and the container is configured to transmit that unique identification with data transmissions to the inventory control hub 104. The inclusion of the unique identification may aid in processing data transmissions from multiple container 102. The container 102 may also include various on-demand support capabilities. In such cases, the container 102 may further include a transmission button that causes the container 102 to send a transmission to the inventory control hub 104 indicating that real-time assistance is needed. The transmission may include the location information of the container 102, and may further include the information about the associated communication device 110. Upon receipt of the transmission, the inventory control hub 104 may communicate to the user through the associated communication device 110. Optionally, in some cases, the container 102 includes a speaker, visual component, or various other features enabling real-time communication with the inventory control hub 104 directly through the container 102 (rather than through the associated communication device 110). Various signage including instructions about various features of the container and/or the auto-injector 208 may also be included.

Referring back to FIG. 1, methods of using the inventory control system 100 to control inventory of the auto-injectors 208 with respect to the container 102 are also provided.

In some examples, a method of inventory control includes starting the controller 108 and various other electronics of the container 102 in a low power state (i.e., hibernation mode) to conserve battery power of the container 102. At predetermined time intervals, the controller 108 exits the low power state and determines medicament inventory levels. In various examples, the predetermined time interval may be a day, a number of hours, or various other suitable time intervals. The controller 108 determines the medicament inventory levels by receiving the sensor data from the sensors 106 and processing that data to determine whether the auto-injector is present or absent from the respective medicament receptacles 206. The controller 108 transmits the inventory level information to the inventory control hub 104, and returns to the low power state for the next time interval.

In other examples, a method of inventory control includes starting the controller 108 and various other electronics of the container 102 in the low power state. If at least one sensor 106 determines that the auto-injector 208 is absent from the corresponding medicament receptacle 206, the controller 108 exits the low power state and send a data transmission to the inventory control hub 104 indicating the absence of the auto-injector 208. In other words, a determination of an absent auto-injector 208 triggers an automatic data transmission from the container 102 to the inventory control hub 104. After sending the data transmission to the inventory control hub 104, the controller 108 returns to the low power state. Optionally, the controller 108 may exit the low power state at predetermined time intervals to send supplemental data transmissions after the initial data transmission.

In various other examples, the controller 108 itself keeps track of inventory levels using one of the above methods, and only transmits data to the inventory control hub 104 after determining that the number of unused auto-injectors 208 is at or below a threshold level. As a non-limiting example, the container 102 may be configured to hold eight auto-injectors 208, and a threshold level may be four unused auto-injectors 208. In this example, the controller 108 may not transmit inventory data to the inventory control hub 104 if one, two, or three auto-injectors 208 have been removed (i.e., seven, six, or five unused auto-injectors 208, respectively, remain in the container 102), but will transmit the inventory data when four auto-injectors 208 are removed (and four auto-injectors 208 remain within the container 102).

Optionally, the inventory control hub 104 uses the inventory data from the container 102 to determine whether a communication with the associated communication device 110 (or the container 102 itself) is needed. For example, the inventory control hub 104 may communicate with the associated communication device 110 if at least one auto-injector 208 is absent from the container 102, the number of auto-injectors 208 is below a threshold level, etc. In various examples, the inventory control hub 104 communicates with the associated communication device 110 upon receipt of the data transmission from the container 102. In other examples, the inventory control hub 104 may delay its communication with the associated communication device 110 by a predetermined time interval (e.g., a day, a number of hours, etc.). In such cases, during the delay, the inventory control hub 104 may determine whether the inventory control hub 104 receives a restocking order for the location 112 (e.g., through the communication device 110 or other communication device) to replace the absent auto-injector 208. The automatic communication between the inventory control hub 104 and the associated communication device 110 may ensure that the users at the location 112 associated with the container 102 are aware that the auto-injector 208 inventory is reduced. The automatic communication between the inventory control hub 104 and the associated communication device 110 may also reduce the processing and stocking time ordinarily needed to order replacement auto-injectors 208.

The above-described aspects are merely possible examples of implementations, merely set forth for a clear understanding of the principles of the present disclosure. Many variations and modifications can be made to the above-described example(s) without departing substantially from the spirit and principles of the present disclosure. All such modifications and variations are included herein within the scope of the present disclosure, and all possible claims to individual aspects or combinations of elements or steps are intended to be supported by the present disclosure. Moreover, although specific terms are employed herein, as well as in the claims that follow, they are used only in a generic and descriptive sense, and not for the purposes of limiting the described invention, nor the claims that follow.

That which is claimed is:

1. An inventory control system comprising:
an inventory control hub; and
a medicament container in communication with the inventory control hub and configured to exchange medicament information with the inventory control hub, the medicament container comprising:
a body defining a container cavity;
a plurality of medicament receptacles, each medicament receptacle configured to accommodate removable storage of an unused auto-injector within the medicament receptacle and prevent storage of a used auto-injector within the medicament receptacle; and
a presence sensor for each medicament receptacle, each presence sensor configured to detect a presence or absence of the unused auto-injector from the corresponding medicament receptacle.

2. The inventory control system of claim 1, wherein the medicament container further comprises a controller in communication with each presence sensor, wherein the controller is configured to remain in a low power state for predetermined time intervals, and wherein after each predetermined time interval, the controller is configured to:
exit the low power state;
receive sensor data from each presence sensor;
determine medicament inventory level information based on the sensor data;
transmit the medicament inventory level information to the inventory control hub; and
return to the low power state and start another predetermined time interval.

3. The inventory control system of claim 1, wherein the medicament container further comprises a controller in communication with each of the presence sensors, wherein the controller is configured to:
enter a low power state;
exit the low power state if at least one of the presence sensors detects an absence of an unused auto-injectors from the corresponding medicament receptacle;
transmit medicament inventory level information to the inventory control hub based on the absence of the unused auto-injector; and
return to the low power state.

4. The inventory control system of claim 1, wherein the presence sensors are selected from the group consisting of mechanical switches, photoelectric sensors, capacitance sensors, optical sensors, pressure sensors, weight sensors, and ultrasonic sensors.

5. The inventory control system of claim 1, wherein the medicament container comprises at least one of:
a transmission button, wherein the transmission button is configured to cause the medicament container to transmit medicament inventory level information to the inventory control hub when selected;
an on-demand communication component enabling real-time communication with the inventory control hub;
a replaceable power source and a visual indicator indicating a status of the replaceable power source; or
a holder for each medicament receptacle.

6. The inventory control system of claim 1, wherein each presence sensor is positioned within the corresponding medicament receptacle.
